(19) United States
(12) Patent Application Publication
(10) Pub. No.: US 2026/0198877 A1
(43) Pub. Date: Jul. 16, 2026

(54) SUPPORT PAD FOR HOLDING X-RAY IMAGE RECEPTORS

(71) Applicant: Kerry Flaherty, Fountain Valley, CA (US)

(72) Inventor: Kerry Flaherty, Fountain Valley, CA (US)

(21) Appl. No.: 19/022,075

(22) Filed: Jan. 15, 2025

(51) Int. Cl. A61B 6/00; A61B 6/42

(52) U.S. Cl. CPC A61B 6/4405; A61B 6/42; A61B 6/4411

(57) ABSTRACT

A support pad for holding X-ray image receptors includes a layer and a plurality of grooves in the layer. The layer has a top surface, a bottom surface, and a perimeter surface connecting the top surface and the bottom surface. The top surface is designed to have a patient placed in a supine position thereon. The bottom surface is designed to be placed on a support structure. Each of the grooves is elongated. Each of the grooves is open at the top surface. Each of the grooves is designed to receive a portion of an X-ray image receptor. Each of the grooves is designed to releasably hold the X-ray image receptor in a vertical direction perpendicular to the top surface.

### (b) CROSS-REFERENCE TO RELATED APPLICATIONS

Not Applicable

### (c) STATEMENT REGARDING FEDERALLY SPONSORED RESEARCH OR

### DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to support pads for hospital gurneys and tables and more particularly pertains to a new support pad for holding X-ray image receptors. In the medical field, X-ray image receptors are plate-shaped structures or cassettes that are placed behind a portion of a body of a patient to be X-rayed. The X-ray machine is placed in front of the portion of the body, such that the portion of the body is between the X-ray machine and the X-ray image receptor. Usually, the patient has to position his or her body in the appropriate location for the X-ray procedure, or the patient has to be positioned by medical staff. If the patient is compromised by age, injury, or illness, then such positioning can be very uncomfortable and difficult, if not hazardous. The new support pad provides a way to X-ray patients in a supine position on a support structure, such as a bed, gurney, or table, without the need to move the patient out of the supine position or off of the support structure. The support pad includes grooves to hold the X-ray image receptors next to the portion of the body to be X-rayed such that the medical staff can perform a lateral X-ray of the portion of the body.

### (2) Description of Related Art Including Information Disclosed Under 37 CFR 1.97 and 1.98

The prior art relates to gurneys and emergency room tables that have space or supports under the bed or pad for insertion of X-ray image receptors or cassettes. The prior art, as best understood, does not disclose a pad to be placed on a gurney or similar that includes grooves in the pad for placement of X-ray image receptors vertically in the pad adjacent portions of the body for lateral X-ray procedures.

### (h) BRIEF SUMMARY OF THE INVENTION

An embodiment of the disclosure meets the needs presented above in a support pad for holding X-ray image receptors generally comprising a layer and a plurality of grooves in the layer. The layer has a top surface, a bottom surface, and a perimeter surface connecting the top surface and the bottom surface. The top surface is designed to have a patient placed in a supine position thereon. The bottom surface is designed to be placed on a support structure. Each of the grooves is elongated. Each of the grooves is open at the top surface. Each of the grooves is designed to receive a portion of an X-ray image receptor. Each of the grooves is designed to releasably hold the X-ray image receptor in a vertical direction perpendicular to the top surface.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of a support pad according to an embodiment of the disclosure.

FIG. 2 is a bottom view of an embodiment of the disclosure.

FIG. 3 is a side view of an embodiment of the disclosure in use.

FIG. 4 is an end view of an embodiment of the disclosure in use.

### (J) DETAILED DESCRIPTION OF THE INVENTION

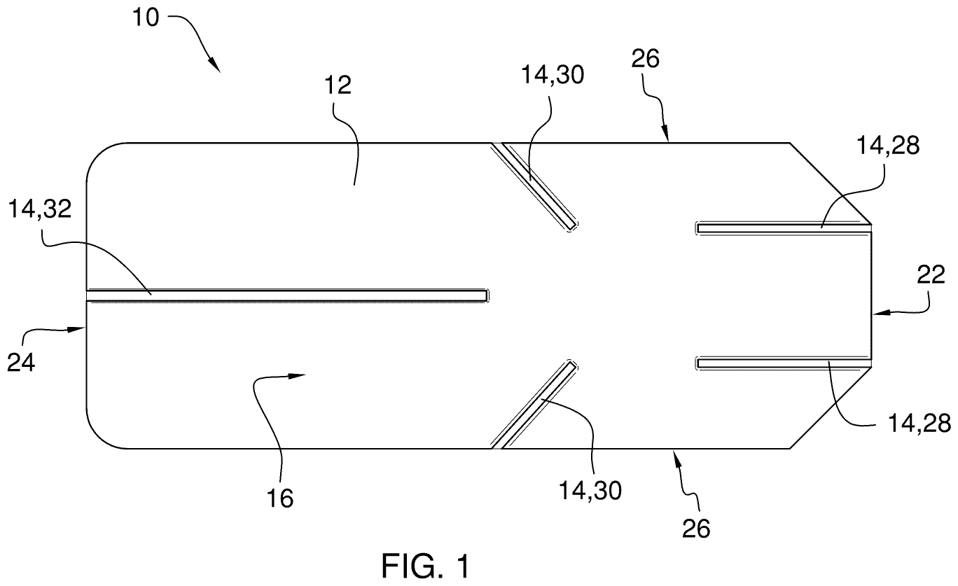

With reference now to the drawings, and in particular to FIGS. 1 through 4 thereof, a new support pad embodying the principles and concepts of an embodiment of the disclosure and generally designated by the reference numeral 10 will be described.

As best illustrated in FIGS. 1 through 4, the support pad 10 generally comprises a layer 12 and a plurality of grooves 14 in the layer 12. The layer 12 has a top surface 16, a bottom surface 18, and a perimeter surface 20 connecting the top surface 16 and the bottom surface 18. The top surface 16 is designed to have a patient 60 placed in a supine position thereon. The bottom surface 18 is designed to be placed on a support structure. Each of the grooves 14 is elongated. Each of the grooves 14 is open at the top surface 16. Each of the grooves 14 is designed to receive a portion of an X-ray image receptor 70. Each of the grooves 14 is designed to releasably hold the X-ray image receptor 70 in a vertical direction perpendicular to the top surface 16.

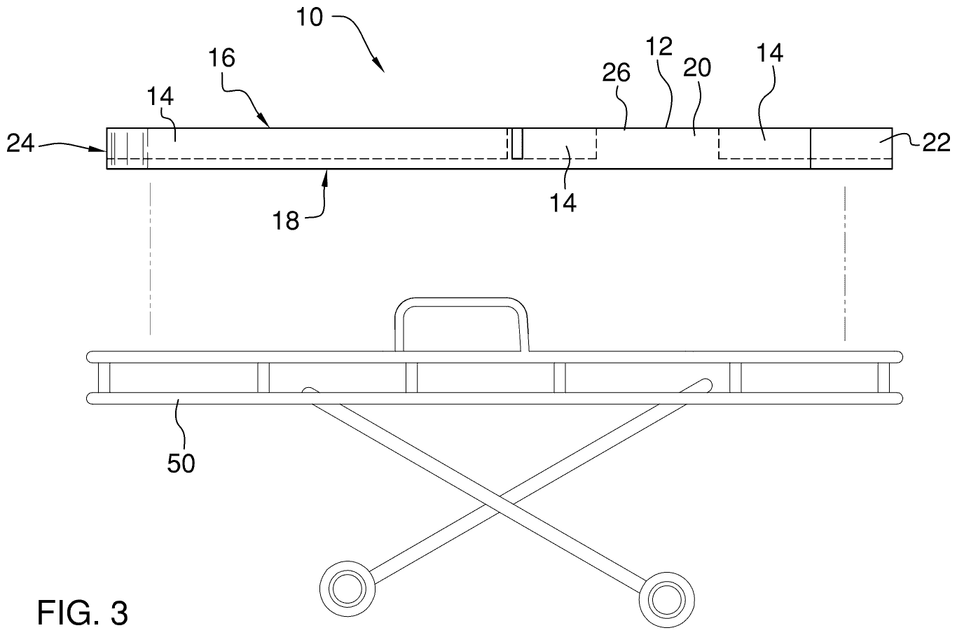

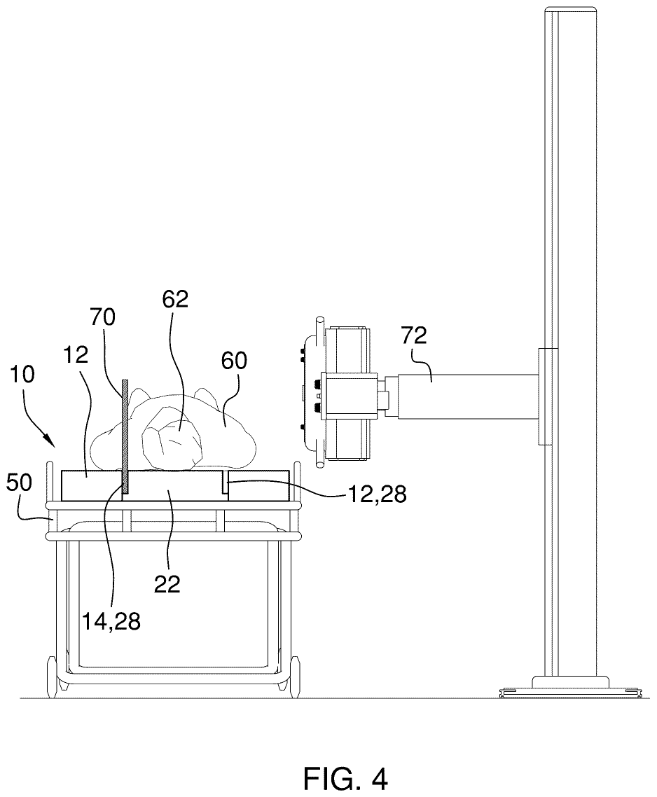

In the exemplary embodiment shown in FIG. 3, each of the grooves 14 have a depth less than a thickness of the layer 12. Consequently, there is material beneath the X-ray image receptor 70 to support and cushion the X-ray image receptor 70, as shown in FIG. 4. However, in another possible embodiment, the grooves 14 have a depth equivalent to the thickness of the layer 12 such that the grooves 14 are open in both the top surface 16 and the bottom surface 18 of the layer 12.

In the exemplary embodiment, each of the grooves 14 is open at the perimeter surface 20 to receive the X-ray image receptor 70 inserted in a direction parallel to the top surface 16. However, in another possible embodiment, each of the grooves 14 does not open in the perimeter surface 20 and is in the form of a pocket or receptacle.

Each of the grooves 14 includes a frictional inner surface to further hold the X-ray image receptor 70. The frictional inner surface could be a coating, such as a rubber or elastomer coating.

In one possible embodiment, the layer 12 is made of a foam material. The layer 12 could also be made of memory foam. The layer 12 is made of material that is sufficiently stiff to hold the X-ray image receptor 70 in a vertical orientation, otherwise the X-ray image receptor 70 could easily fall or tip over. The layer 12 could also be water-impermeable or have a water-impermeable coating to promote cleaning and prevent any liquids from penetrating into the layer 12. In one possible embodiment, a fabric cover could be placed around the layer 12.

The layer 12 should be sized to match the average body size of an adult or pediatric patient 60. In the exemplary embodiment, the layer 12 is rectangular and sized to fit a gurney 50. For example, the layer 12 is at least two inches thick. In addition, the layer 12 is four to seven feet in length and two to four feet in width. These are exemplary measurements and the support pad 10 could be designed to be smaller or larger as desired.

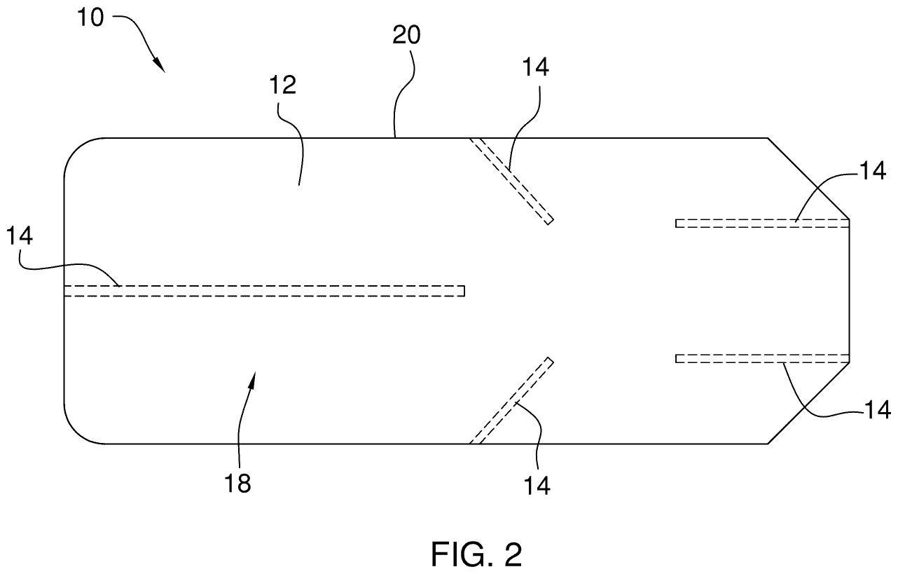

FIGS. 1 and 2 show one possible arrangement of the grooves 14. However, this is an exemplary embodiment and should not be considered as limiting the claims or any embodiments of the invention. The layer 12 could include any number of grooves 14 in any desired arrangement. For example, the grooves 14 could be arranged in a grid or crisscross pattern across the layer 12 to maximize the possible placement positions of the X-ray image receptors 70. In another possible embodiment, the grooves 14 could be arranged in various locations particular to different lateral X-rays.

In FIGS. 1 and 2, the perimeter surface 20 includes a head end surface 22, a foot end surface 24, and a pair of side surfaces 26 positioned perpendicular to and connecting the head end surface 22 and the foot end surface 24. The grooves 14 include a pair of head grooves 28 extending perpendicularly from the head end surface 22. Each of the head grooves 28 is positioned to support the X-ray image receptor 70 to a side of a head 62 of the patient 60. The grooves 14 include a pair of arm grooves 30 extending at an acute angle from a respective one of the side surfaces 26. Each of the arm grooves 30 is positioned to support the X-ray image receptor 70 to a side of an arm of the patient 60. The grooves 14 include a leg groove 32 extending perpendicularly from the foot end surface 24. The leg groove 32 is positioned to support the X-ray image receptor 70 between legs of the patient 60.

FIG. 4 shows the support pad 10 placed on the gurney 50. The X-ray image receptor 70 has been placed in one of the head grooves 28 to the left side of the head 62 of the patient 60. An X-ray machine 72 is placed to the right side of the head 62 of the patient 60 to produce a lateral X-ray of the head 62 of the patient 60. In this manner, the lateral X-ray can be performed without moving the patient 60.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A support pad for holding X-ray image receptors comprising:
a layer having a top surface, a bottom surface, and a perimeter surface connecting said top surface and said bottom surface, said top surface being configured to have a patient placed in a supine position thereon, said bottom surface being configured to be placed on a support structure; and
a plurality of grooves in said layer, each of said grooves being elongated, each of said grooves being open at said top surface, each of said grooves being configured to receive a portion of an X-ray image receptor, each of said grooves being configured to releasably hold the X-ray image receptor in a vertical direction perpendicular to said top surface.

2. The support pad of claim 1, wherein each of said grooves have a depth less than a thickness of said layer.

3. The support pad of claim 1, wherein each of said grooves is open at said perimeter surface to receive the X-ray image receptor inserted in a direction parallel to said top surface.

4. The support pad of claim 1, wherein each of said grooves includes a frictional inner surface to further hold the X-ray image receptor.

5. The support pad of claim 1, wherein said layer is made of a foam material.

6. The support pad of claim 1, wherein said layer is made of memory foam.

7. The support pad of claim 1, wherein said layer is at least two inches thick.

8. The support pad of claim 1, wherein said layer is four to seven feet in length and two to four feet in width.

9. The support pad of claim 1, wherein said perimeter surface includes a head end surface, a foot end surface, and a pair of side surfaces positioned perpendicular to and connecting said head end surface and said foot end surface.

10. The support pad of claim 9, wherein said grooves include a pair of head grooves extending perpendicularly from said head end surface, each of said head grooves is positioned to support the X-ray image receptor to a side of a head of the patient.

11. The support pad of claim 9, wherein said grooves include a pair of arm grooves extending at an acute angle from a respective one of said side surfaces, each of said arm grooves is positioned to support the X-ray image receptor to a side of an arm of the patient.

12. The support pad of claim 9, wherein said grooves include a leg groove extending perpendicularly from said foot end surface, said leg groove is positioned to support the X-ray image receptor between legs of the patient.

13. The support pad of claim 9, wherein said grooves include:
a pair of head grooves extending perpendicularly from said head end surface, each of said head grooves is positioned to support the X-ray image receptor to a side of a head of the patient;
a pair of arm grooves extending at an acute angle from a respective one of said side surfaces, each of said arm grooves is positioned to support the X-ray image receptor to a side of an arm of the patient; and
a leg groove extending perpendicularly from said foot end surface, said leg groove is positioned to support the X-ray image receptor between legs of the patient.

14. The support pad of claim 13, wherein each of said grooves have a depth less than a thickness of said layer.

15. The support pad of claim 14, wherein each of said grooves is open at said perimeter surface to receive the X-ray image receptor inserted in a direction parallel to said top surface.

16. The support pad of claim 15, wherein each of said grooves includes a frictional inner surface to further hold the X-ray image receptor.

17. The support pad of claim 16, wherein said layer is made of a foam material.

18. The support pad of claim 17, wherein said layer is made of memory foam.

19. The support pad of claim 18, wherein said layer is at least two inches thick.

20. The support pad of claim 19, wherein said layer is four to seven feet in length and two to four feet in width.
